(19) United States
(12) Patent Application Publication
(10) Pub. No.: US 2006/0068491 A1
(43) Pub. Date: Mar. 30, 2006

(54) Microdevice for performing method of separating and purifying nucleic acid

(75) Inventors: Yoshihiko Makino, Asaka-shi (JP); Yoshiki Sakaino, Asaka-shi (JP); Yukio Sudo, Minami-Ashigara-shi (JP); Yoshihiko Abe, Asaka-shi (JP)

Correspondence Address: BIRCH STEWART KOLASCH & BIRCH, PO BOX 747, FALLS CHURCH, VA, 22040-0747, US

(73) Assignee: Fuji Photo Film Co., Ltd.

(21) Appl. No.: 11/227,245

(22) Filed: Sep. 16, 2005

(30) Foreign Application Priority Data
Sep. 24, 2004 (JP) P.2004-278070

(51) Int. Cl. C12M 1/34; C07H 21/04

(52) U.S. Cl. 435/287.2; 536/25.4

(57) ABSTRACT

A microdevice for performing a method for separating and purifying a nucleic acid, the microdevice comprising: at least one opening; and at least one channel for passing a sample solution, wherein the method comprises: (A) a step of bringing a nucleic acid-containing sample solution into contact with a nucleic acid-adsorbing support having a function of adsorbing a nucleic acid; (B) a step of washing the nucleic acid-adsorbing support with a washing solution in a state of a nucleic acid being adsorbed to the support; and (C) a step of desorbing the nucleic acid from the nucleic acid-adsorbing support by a recovering solution, thereby purifying the nucleic acid; an apparatus for utilizing the microdevice; and a reagent kit for use in the microdevice.

### BACKGROUND OF THE INVENTION

1. Field of the Invention

The present invention relates to a microdevice for performing a method of separating and purifying a nucleic acid. More specifically, the present invention relates to a microdevice for performing a method of separating and purifying a nucleic acid, comprising at least one or more opening and one or more channel for passing a nucleic acid-containing sample solution. Still more specifically, the present invention relates to a microdevice where a nucleic acid-adsorbing support having a function of adsorbing a nucleic acid is received in the microdevice, where the channel is made of a nucleic acid-adsorbing support, or where a nucleic acid-adsorbing structure is present as a nucleic acid-adsorbing support in the channel.

2. Description of the Related Art

The nucleic acid is being used in various forms in various fields. For example, in the region of recombinant nucleic acid technology, the nucleic acid is required to be used in the form of a probe, a genome nucleic acid or a plasmid nucleic acid.

Also in the diagnosis field, the nucleic acid is being used in various forms for various purposes. For example, the nucleic acid probe is commonly used for the detection and diagnosis of a human pathogen. Similarly, the nucleic acid is used for the detection of a genetic disorder. In addition, the nucleic acid is used for the detection of a food contaminant. Furthermore, it is popularized to use a nucleic acid in the localization, identification and isolation of an interesting nucleic acid for various purposes in the process from the production of a genetic map to the cloning and expression of recombination.

In many cases, the nucleic acid is available in a very small amount and the operation for the isolation and purification thereof is cumbersome and takes much time. This cumbersome operation which often consumes much time readily leads to loss of the nucleic acid. In the case of purifying a nucleic acid from a sample obtained by using a culture of serum, urine or bacteria, there is additionally a danger of causing contamination or false-positive result.

One of widely known separation and purification methods is a method of adsorbing a nucleic acid to a solid phase of silicon dioxide, silica polymer, magnesium silicate or the like, and subsequently performing operations such as washing and desorption to effect the separation and purification (see, for example, JP-B-7-51065 (the term “JP-B” as used herein means an “examined Japanese patent publication”)). This method exhibits excellent separation performance but is not satisfied in view of easiness, swiftness and suitability for automation. In addition, the device and apparatus used for this method are unsuited for automation and downsizing. Furthermore, the device and apparatus, particularly, adsorption medium, can be hardly mass-produced in industry with the same performance and also have a problem that, for example, the handling is inconvenient and the processing into various shapes is difficult.

As one of the methods for easily and efficiently separating and purifying a nucleic acid, there has been proposed a method where a nucleic acid is adsorbed to and desorbed from a solid phase- comprising an organic polymer having a hydroxyl group on the surface thereof by using a solution of adsorbing a nucleic acid to a solid phase and a solution of desorbing a nucleic acid from a solid phase, respectively, thereby separating and purifying the nucleic acid (see, JP-A-2003-128691 (the term “JP-A” as used herein means an “unexamined published Japanese patent application”)), but more improvement is demanded.

Other examples of the conventionally known method for separating and purifying a nucleic acid include those using centrifugation, using magnetic beads or using a porous membrane. Also, an apparatus for separating and purifying a nucleic acid by utilizing such a method has been proposed. For example, with respect to the apparatus for separating and purifying a nucleic acid by using a porous membrane, there has been proposed an automatic apparatus where after many porous membrane tubes each receiving a porous membrane are set in a rack, a nucleic acid-containing sample solution is injected into each tube, the peripheral of the bottom of the rack is tightly closed by an air chamber through a seal material, the inside is depressurized, all porous membrane tubes are at the same time sucked from the discharge side to allow for passing of the sample solution and adsorption of a nucleic acid to the porous membrane, a washing solution and a recovering solution are injected, and the tubes are again depressurized and sucked to effect washing and desorption (see, for example, Japanese Patent No. 2832586).

On the other hand, a reaction apparatus having a fine channel, that is, a microscale channel, is generically called a “microreactor” in general and this is making a great progress in recent years (see, W. Ehrfeld, V. Hessel and H. Lowe, Microreactor, 1st ed., WILEY-VCH (2000)).

The microdevice, so-called microreactor, is used as a microfluid device comprising a member in which, for example, a fine channel (mainly having an equivalent diameter of 1 mm or less) or a structure connected to the channel, such as reaction tank, electrophoresis column and membrane separation mechanism, is appropriately formed. This microfluid device has a capillary channel in the inside and is expected to be usable as a microreaction device (micro-reactor) for chemical or biochemical use, for example, as a microanalysis device (e.g., integrated DNA analysis device, microelectrophoresis device, microchromatography device), a microdevice for the preparation of an assay sample in mass spectrum, liquid chromatography or the like, a device for physicochemical treatment such as extraction, membrane separation and dialysis, or a spotter for the production of a microarray.

### SUMMARY OF THE INVENTION

Accordingly, an object of the present invention is to provide a microdevice for easily and swiftly separating and purifying a nucleic acid from a nucleic acid-containing sample solution while maintaining the yield and purity in conventional methods for separating a nucleic acid.
(1) A microdevice for performing a method for separating and purifying a nucleic acid, the microdevice comprising:
at least one opening; and
at least one channel for passing a sample solution,
wherein the method comprises:
   (A) a step of bringing a nucleic acid-containing sample solution into contact with a nucleic acid-adsorbing support having a function of adsorbing a nucleic acid;
   (B) a step of washing the nucleic acid-adsorbing support with a washing solution in a state of a nucleic acid being adsorbed to the support; and
   (C) a step of desorbing the nucleic acid from the nucleic acid-adsorbing support by a recovering solution, thereby purifying the nucleic acid.
(2) The microdevice as described in (1) above,
wherein the method for separating and purifying a nucleic acid by utilizing the microdevice comprises a pretreatment step of:
mixing a test sample and a nucleic acid-solubilizing reagent, so as to obtain a mixture; and
uniformizing the mixture to obtain a nucleic acid-containing sample solution, and
the microdevice further comprises a mechanism of performing the pretreatment step.
(3) The microdevice as described in (1) or (2) above,
wherein the channel has a width of 1 to 3,000 μm.
(4) The microdevice as described in any of (1) to (3) above,
wherein the microdevice receives a nucleic acid-adsorbing porous membrane as the nucleic acid-adsorbing support.
(5) The microdevice as described in any of (1) to (3) above,
wherein the microdevice receives a nucleic acid-adsorbing bead as the nucleic acid-adsorbing support.
(6) The microdevice as described in any of (1) to (3) above,
wherein the channel comprises a nucleic acid-adsorbing support.
(7) The microdevice as described in any of (1) to (3) above,
wherein the channel has a nucleic acid-adsorbing structure as the nucleic acid-adsorbing support in the channel.
(8) The microdevice as described in any of (1) to (7) above,
wherein the sample solution is a solution resulting from adding a water-soluble organic solvent to a solution obtained by treating a test sample with a nucleic acid-solubilizing reagent.
(9) The microdevice as described in any of (1) to (8) above,
wherein the nucleic acid-solubilizing reagent is a solution containing at least one of a chaotropic salt, a surfactant, a protease, an antifoaming agent and a nucleic acid stabilizer.
(10) The microdevice as described in any of (1) to (9) above,
wherein the washing solution is a solution containing at least one of methanol, ethanol, propanol or an isomer thereof, and butanol or an isomer thereof in an amount of 20 to 100 weight %.
(11) The microdevice as described in any of (1) to (10) above,
wherein the recovering solution is a solution having a salt concentration of 0.5 mol/L or less.
(12) An apparatus for utilizing a microdevice as described in any of (1) to (11) above.
(13) A reagent kit for use in a microdevice as described in any of (1) to (11) above.

### BRIEF DESCRIPTION OF THE DRAWINGS

FIGS. 1A to 1C are views for describing the process of producing a microdevice using a porous membrane; FIG. 1A is a perspective view of a PDMS plate in which a channel is formed; FIG. 1B is a front view when a porous membrane is sandwiched by two chips; and FIG. 1C is a schematic view showing the cross section of the device;

FIG. 2 is an explanatory view for describing the passage of liquids by the effect of a pressure applied from a pump;

FIG. 3A is an explanatory view of a microdevice using beads; and FIG. 3B is a view for describing the mechanism of preventing the beads from flowing out at the end of the channel;

FIG. 4A is a plan view of a device with a channel pattern; and FIG. 4B is an explanatory view of the cross section of the channel;

FIG. 5A is an explanatory view of a microdevice having structures in the channel; and FIG. 5B is a schematic view showing the A-A cross section of the channel in which nanosize pillars are provided; and

FIG. 6 is an electrophoretogram showing results after amplification by PCR, in comparison and contrast.

### DETAILED DESCRIPTION OF THE INVENTION

<Microdevice>

The microdevice for performing a method for separating and purifying a nucleic acid of the present invention is a microdevice characterized by having at least one or more opening and one or more channel for passing a sample solution.

In the present invention, the microdevice means an apparatus having a channel (also called a flow path) with an equivalent diameter of 1 mm or less.

The equivalent diameter as used in the present invention is also called an equivalent (diameter) size and this term is generally used in the field of mechanical engineering. When a circular tube equivalent to a pipeline (in the present invention, the channel) having an arbitrary cross-sectional shape is imagined, the diameter of the equivalent circular tube is called an equivalent diameter, and the equivalent diameter deq is defined as deq=4 A/p by using a cross-sectional area A of the pipeline and a wetted perimeter length (circumferential length) p of the pipeline. When this is applied to a circular tube, the equivalent diameter agrees with the diameter of the circular tube. The equivalent diameter is used for estimating the fluidity or heat transfer characteristics of the pipeline based on the data of the equivalent circular tube, and this diameter represents a spatial scale (representative length) of a phenomenon. The equivalent diameter is deq=4a²/4 a=a in the case of a regular square tube with one side of a and deq=2h in the case of a flow between parallel flat plates with a path height of h. Details thereon are described in Kikai Kogaku Jiten (Mechanical Engineering Dictionary), compiled by The Japan Society of Mechanical Engineers, Maruzen (1997).

The equivalent diameter of the channel for use in the present invention is 1 mm or less, preferably from 10 to 500 μm, more preferably from 20 to 300 μm.

The length of the channel is not particularly limited but is preferably from 1 to 10,000 mm, more preferably from 5 to 100 mm.

The width of the channel for use in the present invention is preferably from 1 to 3,000 μm, more preferably from 10 to 2,000 μm, still more preferably from 50 to 1,000 μm. With a channel width in this range, the sample solution less receives resistance from the channel wall to decrease in the flowability, and the amount of the sample solution can be advantageously set to be small.

In the present invention, the microdevice and the channel can be produced on a solid substrate by a fine processing technique.

Examples of the material used as the solid substrate include metal, silicon, Teflon, glass, ceramic and plastic. Among these, metal, silicon, polydimethylsiloxane (PDMS), tetrafluoroethylene, glass and ceramic are preferred in view of heat resistance, pressure resistance, solvent resistance and light transmittance, and PDMS is more preferred.

Examples of the fine processing technique for producing the channel include the methods described in Microreactor-Shin Jidai no Gosei Gijutsu-(Microreactor-Synthesis Technology of New Era-) (supervisor: Junichi Yoshida, Professor of Graduate School of Engineering at Kyoto University; issued by CMC (2003)) and Bisai Kakou Gijutsu, Oyo Hen—Photonics•Electronics•Mechatronics eno Oyo- (Fine Processing Technology, Application—Application to Photonics•Electronics•Mechatronics-) (compiled by Gyoji Iinkai of SPSJ, issued by NTS (2003)).

Representative examples of the method include an LIGA technique using X-ray lithography, a high-aspect-ratio photolithography method using EPON SU-8, a microdischarge processing method (μ-EDM), a high-aspect-ratio processing method for silicon by deep RIE, a hot emboss processing method, a stereolithography method, a laser processing method, an ion beam processing method and a mechanical microcutting method using a microtool formed of a hard material such as diamond. These techniques may be used individually or in combination. Among these fine processing techniques, preferred are an LIGA technique using X-ray lithography, a high-aspect-ratio photo-lithography method using EPON SU-8, a microdischarge processing method (μ-EDM) and a mechanical microcutting method.

The channel for use in the present invention can be produced by casting a resin in a mold comprising a pattern formed on a silicon wafer with use of a photoresist and then solidifying the resin (molding method). In the molding method, a silicon resin as represented by polydimethylsiloxane (PDMS) or a derivative thereof can be used.

In fabricating the microdevice of the present invention, a junction technique can be used. The normal junction technique is roughly classified into solid phase junction and liquid phase junction. As for the junction method commonly employed, representative examples of the solid phase junction method include pressure welding and diffusion junction, and representative examples of the liquid phase junction include welding, eutectic bonding, soldering and adhesion.

Furthermore, the fabrication is preferably performed by using a highly precise junction method of keeping the dimensional accuracy without causing fracture of a microstructure such as channel due to deterioration or large deformation of a material under high-temperature heating, and examples of the technique therefor include silicon direct bonding, anodic bonding, surface activation bonding, direct junction using hydrogen bonding, junction using an aqueous HF solution, Au—Si eutectic bonding and void-free adhesion.

The sample solution transfers in the channel. The sample solution, namely, a fluid in the channel is preferably handled by a continuous flowing system, a liquid droplet (liquid plug) system, a driving system or the like or by using a capillary phenomenon.

In controlling a fluid by the continuous flowing system, the inside of the channel in the microdevice must be entirely filled with a fluid, and the fluid as a whole is generally driven by a pressure source prepared outside, such as syringe pump. When the continuous flowing system is employed, a control system can be realized by a relatively simple and easy set-up.

In the liquid droplet (liquid plug) system, liquid droplets partitioned by air are transported inside the device or in the channel reaching the device, and individual liquid droplets are driven by the air pressure. In the liquid droplet system, for example, a vent structure of allowing an air between the liquid droplet and the channel wall or between liquid droplets to escape outside according to the necessity, or a valve structure for keeping the pressure in the branched channel to be independent of other portions must be provided inside the device system. Furthermore, since the liquid droplets are operated by controlling the pressure difference, a pressure control system comprising a pressure source and a changeover valve must be exteriorly constructed. The liquid droplet system is advantageous in that a multi-stage operation of individually operating a plurality of liquid droplets and sequentially performing several reactions can be performed and the latitude in the system construction is broadened.

The driving system widely employed in general is an electrical driving system of applying a high voltage to both ends of the channel to generate an electroosmosis flow and transporting a fluid by the flow, a pressure driving system of exteriorly preparing a pressure source and transporting a fluid by applying a pressure, or a driving system utilizing a capillary phenomenon.

It is known that in the electrical driving system, the fluid behaves to give a flat distribution of the flow rate profile within the cross section of the channel, whereas in the pressure driving system, the fluid behaves to give a hyperbolic distribution of the flow rate profile, namely, high flow rate in the channel center part and low flow rate in the wall surface part. For the purpose of transporting the fluid while keeping the shape of sample plug or the like, the electrical driving system is preferred.

In the electrical driving system, the inside of the channel must be filled with a fluid, that is, the mode is a continuous flowing system. The fluid can be operated by electrical control and therefore, a relatively complicated treatment of, for example, continuously changing the mixing ratio of two kinds of solutions and creating a temporal concentration gradient can be performed.

In the pressure driving system, the control can be performed without any effect of the electrical property peculiar to the fluid. Since secondary effects such as heat generation or electrolysis need not be taken account of and the substrate is scarcely affected, the application range of this system is broad. In the pressure driving system, a pressure source must be exteriorly prepared.

In the present invention, the system of transporting the test sample can be appropriately selected in accordance with the kind of test sample or sample solution used for the separation and purification of a nucleic acid, the nucleic acid-adsorbing support or microdevice used, and the like. Among these systems, preferred are a liquid droplet (liquid plug) system and a driving system utilizing a capillary phenomenon, more preferred is a liquid droplet (liquid plug) system in which the air pressure is a negative pressure, and still more preferred is a liquid droplet system in which the negative pressure is created by the suction of air.

<Nucleic Acid-Adsorbing Support>

The nucleic acid-adsorbing support (hereinafter sometimes simply referred to as a “support”) for use in the present invention is characterized by having a function of adsorbing a nucleic acid. The term “having a function of adsorbing a nucleic acid” means that the surface has a function of allowing for adsorption of a nucleic acid by the effect of an interaction substantially not involving ion bonding. This denotes no occurrence of “ionization” of the support under the conditions in use and implies that a nucleic acid and the support attract each other as a result of change in the polarity of the environment. By virtue of this function, a nucleic acid can be isolated and purified with excellent separation performance and good washing efficiency.

The support is received in the microdevice. Also, the channel in the microdevice may comprise the nucleic acid-adsorbing support.

The nucleic acid-adsorbing support is preferably a support having a hydrophilic group, and it is presumed that when the polarity of the environment is changed, a nucleic acid and the hydrophilic group on the support surface are caused to attract each other.

{Hydrophilic Group}

The hydrophilic group indicates a polar group (atomic group) capable of interacting with water, and all groups (atomic groups) participating in the adsorption of a nucleic acid come under the hydrophilic group. The hydrophilic group is preferably a hydrophilic group having a moderate strength of interaction with water (see, “Group Having Not So Strong Hydrophilicity” in “Hydrophilic Group” of Encyclopaedia Chimica, Kyoritsu Shuppan), and examples thereof include a hydroxyl group, a carboxyl group, a cyano group and an oxyethylene group. Among these, a hydroxyl group is preferred.

Here, the “support having a hydrophilic group” means that the nucleic acid-adsorbing support has a hydrophilic group or a hydrophilic group is introduced into the support by treating or coating the material constituting the support. Also, the “channel in the microdevice comprises a nucleic acid-adsorbing support having a hydrophilic group” means that the material constituting the channel has a hydrophilic group or a hydrophilic group is introduced by treating or coating the material constituting the channel.

The support or the material constituting the support may be an organic material or an inorganic material. For example, the material constituting the support may be an organic material having a hydrophilic group, or an organic material not having a hydrophilic group may be treated to introduce a hydrophilic group and used as the support or may be coated with a material having a hydrophilic group to introduce a hydrophilic group and used as the support. Also, the material constituting the support may be an inorganic material having a hydrophilic group, or an inorganic material not having a hydrophilic group may be treated to introduce a hydrophilic group and used as the support or may be coated with a material having a hydrophilic group to introduce a hydrophilic group and used as the support. In view of easiness of processing, the support or the material constituting the support is preferably an organic material such as organic polymer.

The material having a hydrophilic group includes an organic material having a hydroxyl group. Examples of the organic material having a hydroxyl group include a substance formed of a polyhydroxyethylacrylic acid, a polyhydroxyethylmethacrylic acid, a polyvinyl alcohol, a polyoxyethylene, an acetylcellulose, or a mixture of acetylcelluloses different from each other in acetyl value. In particular, an organic material having a polysaccharide structure can be preferably used.

Examples of the organic material having a hydroxyl group, which can be preferably used, include an organic polymer comprising a mixture of a cellulose and an ester compound of a cellulose derivative. Examples of the mixture of cellulose derivatives different from each other in ester value, which can be preferably used, include a mixture of triester cellulose and diester cellulose, a mixture of triester cellulose and monoester cellulose, a mixture of triester cellulose, diester cellulose and monoester cellulose, and a mixture of diester cellulose and monoester cellulose.

More preferred examples of the organic material having a hydroxyl group include acetylcelluloses different from each other in acetyl value and a saponified product thereof described in JP-A-2003-128691. The saponified product of acetylcellulose is obtained by saponifying a mixture of acetylcelluloses different from each other in acetyl value. A saponified product of a triacetylcellulose and diacetylcellulose mixture, a saponified product of a triacetylcellulose and diacetylcellulose mixture, a saponified product of a triacetylcellulose and monoacetylcellulose mixture, a saponified product of a triacetylcellulose, diacetylcellulose and monoacetylcellulose mixture, and a saponified product of a diacetylcellulose and monoacetylcellulose mixture may also be preferably used. Among these, a saponified product of a triacetylcellulose and diacetylcellulose mixture is more preferred. The mixing ratio (by weight) in the triacetylcellulose and diacetylcellulose mixture is preferably from 99:1 to 1:99. The mixing ratio in the triacetylcellulose and diacetylcellulose mixture is more preferably from 90:10 to 50:50 and in this case, the amount (density) of a hydroxyl group on the solid phase surface can be controlled by the degree of saponification (saponification ratio). In order to elevate the separation efficiency of a nucleic acid, the amount (density) of a hydroxyl group is preferably larger. The saponification ratio (surface saponification ratio) of the organic material obtained by saponification is preferably from 5 to 100%, more preferably from 10 to 100%.

Herein, the saponification treatment means that acetyl cellulose comes in contact with saponification treatment solution (e.g., Sodium hydroxide solution). As a result, the saponification treatment solution contacted ester group of ester derivative of acetyl cellulose is hydrolyzed, and a hydroxyl group is introduced to form regenerated cellulose. Thereby the prepared regenerated cellulose is different in crystalline form from the original cellulose. In order to change the surface saponification degree, saponification treatment is conducted having changed the concentration or treating time of sodium hydroxide or potassium hydroxide.

A method for introducing a hydrophilic group comprising organic material not having a hydrophilic group is to bond a graft polymer chain having a hydrophilic group in inner polymer strand or a side chain to a support or material to form microdevice.

A method for bonding a graft polymer chain to an organic material includes two methods such as a method for chemically bonding with graft polymer chain, and a method for polymerizing a compound having a double bond capable of polymerization as a starter to form graft polymer chain.

Firstly, in the method in which the solid phase and graft polymer chain are chemically bonded, a polymer having a functional group capable of reacting with the support or material to form microdevice in the terminus or side chain of the polymer is used, and they are grafted through a chemical reaction of this functional group with a functional group of the support or material to form microdevice. The functional group capable of reacting with the support or material to form microdevice is not particularly limited with the proviso that it can react with a functional group of the support or material to form microdevice, and its examples include a silane coupling group such as alkoxysilane, isocyanate group, amino group, hydroxyl group, carboxyl group, sulfonate group, phosphate group, epoxy group, allyl group, methacryloyl group, acryloyl group and the like.

Examples of the compound particularly useful as the polymer having a reactive functional group in the terminus or side chain of the polymer include a polymer having trialkoxysilyl group in the polymer terminus, a polymer having amino group in the polymer terminus, a polymer having carboxyl group in the polymer terminus, a polymer having epoxy group in the polymer terminus and a polymer having isocyanate group in the polymer terminus. The polymer to be used in this case is not particularly limited with the proviso that it has a hydrophilic group which is concerned in the adsorption of nucleic acid, and its illustrative examples include polyhydroxyethyl acrylic acid, polyhydroxyethyl methacrylic acid and salts thereof, polyvinyl alcohol, polyvinyl pyrrolidone, polyacrylic acid, polymethacrylic acid and salts thereof, polyoxyethylene and the like.

The method in which a compound having a polymerizable double bond is made into a graft polymer chain by polymerizing it using the solid phase as the starting point is generally called surface graft polymerization. The surface graft polymerization method means a method in which an active species is provided on the base material surface by plasma irradiation, light irradiation, heating or the like method, and a polymerizable compound having double bond arranged in contact with a solid phase is linked to the solid phase by polymerization.

It is necessary that the compound useful for forming a graft polymer chain linked to the base material has both of two characteristics of having a polymerizable double bond and having a hydrophilic group which is concerned in the adsorption of nucleic acid. As such a compound, any one of the polymers, oligomers and monomers having a hydrophilic group can be used with the proviso that it has a double bond in the molecule. Particularly useful compound is a monomer having a hydrophilic group.

As illustrative examples of the particularly useful monomer having a hydrophilic group, the following monomers can be cited. For example, 2-hydroxyethyl acrylate, 2-hydroxyethyl methacrylate, glycerol monomethacrylate and the like hydroxyl group-containing monomers can be used particularly suitably. In addition, acrylic acid, methacrylic acid and the like carboxyl group-containing monomers or alkali metal salts and amine salts thereof can also be used suitably.

As another method for introducing a hydrophilic group into an organic material having no hydrophilic group, a material having a hydrophilic group can be coated. The material to be used in the coating is not particularly limited with the proviso that it has a hydrophilic group which is concerned in the adsorption of nucleic acid, but is preferably a polymer of an organic material from the viewpoint of easy handling. Examples of the polymer include polyhydroxyethyl acrylate, polyhydroxyethyl methacrylate and salts thereof, polyvinyl alcohol, polyvinyl pyrrolidone, polyacrylic acid, polymethacrylic acid and salts thereof, polyoxyethylene, acetyl cellulose, a mixture of acetyl celluloses having different acetyl values and the like, but a polymer having a polysaccharide structure is desirable.

Alternatively, after coating an organic material having no hydrophilic group with a cellulose derivative or a mixture of cellulose derivatives, the coated cellulose derivative or the coated mixture of cellulose derivatives can be treated with saponification. The method of the saponification can be achieved by contacting with an alkaline aqueous solution as mentioned above. In that case, the saponification ratio is preferably from 5% or more to 100% or less. The saponification ratio is more preferably from 10% or more to 100% or less.

As the inorganic material having a hydrophilic group, a support containing a silica compound can be exemplified. As the support containing a silica compound, a glass filter, a silica bead or colloidal silica can be exemplified. These are, as mentioned below, used to be received in the channel. Further, the colloidal silica can be used by coating the surface of the channel. As the method for coating the colloidal silica to the surface of the channel, the method in which the channel is filled with the colloidal silica solution, and heat (for example, 65° C./2 h) is exemplified.

Also can be exemplified is a porous silica thin membrane described in Japanese Patent No. 3,058,3442. This porous silica thin membrane can be prepared by spreading a developing solution of a cationic amphipathic substance having an ability to form a bimolecular membrane on a base material, preparing multi-layered bimolecular thin membranes of the amphipathic substance by removing the solvent from the liquid membrane on the base material, allowing the multi-layered bimolecular thin membranes to contact with a solution containing a silica compound, and then extracting and removing the aforementioned multi-layered bimolecular thin membranes.

Regarding the method for introducing a hydrophilic group into an inorganic material having no hydrophilic group, there are a method in which the solid phase and a graft polymer chain are chemically bonded and a method in which a graft polymer chain is polymerized using a hydrophilic group-containing monomer having a double bond in the molecule, using the solid phase as the starting point.

When the inorganic material and graft polymer chain are attached by chemical bonding, a functional group capable of reacting with a terminal functional group of the graft polymer chain is introduced into an inorganic material, and the graft polymer chain is chemically bonded thereto. Also, when a graft polymer chain is polymerized using a hydrophilic group-containing monomer having a double bond in the molecule and using the solid phase as the starting point, a functional group which becomes the starting point in polymerizing the double bond-containing compound is inserted into the inorganic material.

As the graft polymer having a hydrophilic group and hydrophilic group-containing monomer having a double bond in the molecule, the aforementioned graft polymer having a hydrophilic group and hydrophilic group-containing monomer having a double bond in the molecule, described in the foregoing regarding the method for introducing a hydrophilic group into an organic material having no hydrophilic group, can be suitably use.

Another method for introducing a hydrophilic group to inorganic material not having a hydrophilic group is to coat a material having a hydrophilic group thereon. Materials used in coating are not limited as long as the hydrophilic group participates in the adsorption of nucleic acid, but for easy workability, a polymer of organic material is preferred. Examples of polymer include polyhydroxyethylacrylate, polyhydroxyethylmethacrylate and their salts, polyvinyl alcohol, polyvinylpyrrolidone, polyacrylate, polymethacrylate and their salts, polyoxyethylene, acetyl cellulose, and a mixture of acetyl celluloses which are different in acetyl value from each other.

After the inorganic material not having a hydrophilic group is coated with a cellulose derivative or a mixture of cellulose derivatives, the cellulose derivative coated or the mixture of cellulose derivatives coated may be saponified. The saponification can be effected by the contact with an alkaline aqueous solution, similarly to the above. In this case, the saponification ratio is preferably from 5 to 100%, more preferably from 10 to 100%.

Examples of the inorganic material not having a hydrophilic group including aluminum and the like metals, glass, cement, pottery and the like ceramics, or a porous membrane fabricated by stepping new ceramics, silicon, active charcoal, etc.

{Form of Support}

The form of the nucleic acid-adsorbing support is not particularly limited and may be, for example, either sheet or bead. The support is received in the microdevice. Also, the channel of the microdevice may be made of the nucleic acid-adsorbing support.

Preferred embodiments are described below, but the present invention is not limited thereto.

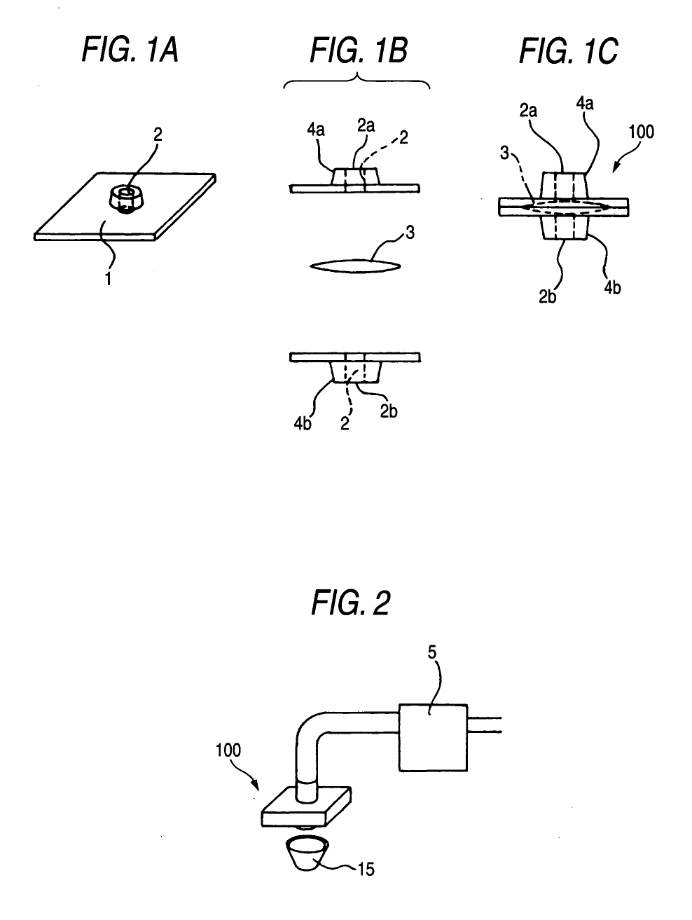

The case where the nucleic acid-adsorbing support has a sheet form is described below. Examples of the sheet form include a fabric form such as woven fabric and knitted fabric, a non-woven fabric form, a paper form, and a shape formed by planarly casting a polymer, such as film. A liquid-passing sheet is preferred in view of recovery efficiency, and a porous membrane or cloth is preferred. A porous membrane (that is, a nucleic acid-adsorbing porous membrane) is more preferred in view of production stability among lots or easy incorporation into the microdevice. The thickness of the sheet is preferably 1,000 μm or less, more preferably 500 μm or less. Within this range, the volume of fluid held in the sheet can be prevented from increasing and the yield can be advantageously maintained. In the case of packing the sheet-form nucleic acid-adsorbing support in the microdevice, that is, disposing the support in the channel of the microdevice, it is preferred from the standpoint of facilitating the production that, as shown in FIGS. 1A to 1C, the channel 2 is designed to penetrate the microdevice 100 and upper and lower two chips 4a and 4b constituting the microdevice are bonded to sandwich the sheet 3. The bonding method may be the above-described solid phase junction or liquid phase junction, and the method therefor is not limited.

The case where the nucleic acid-adsorbing support is a nucleic acid-adsorbing bead is described below. The beads are not always required to have a spherical form and also, need not be necessarily uniform in the shape. In the case of extracting a nucleic acid from blood, cell or the like, the beads preferably have a spherical form and are uniform in the size from the standpoint of decreasing the residual fluid in the channel within the microchip. The average particle diameter of the beads is preferably from 0.1 to 500 μm, more preferably from 1 to 100 μm. The bead itself may be made of an organic polymer of allowing for adsorption of a nucleic acid by the effect of a weak interaction which does not involve the above-described ion bonding, or an organic polymer having such an interaction may be physically or chemically bonded to the bead surface.

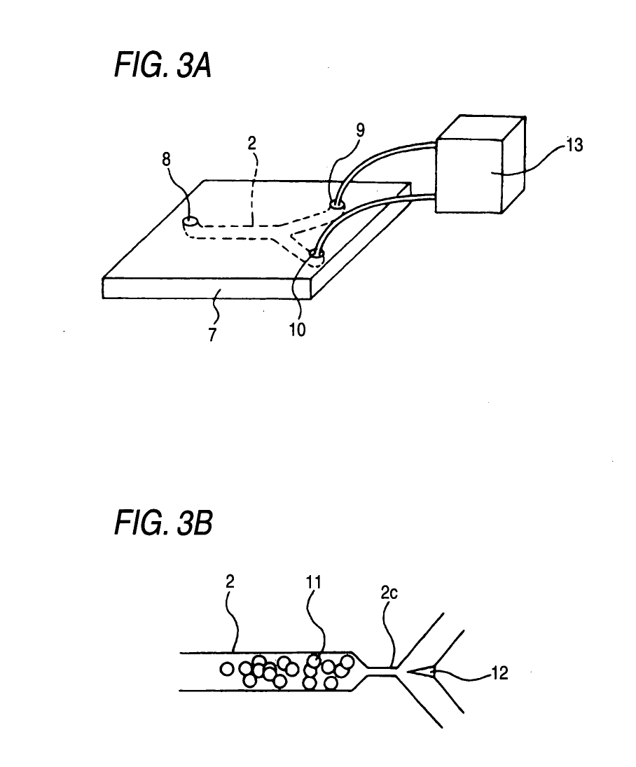

The beads are, as shown in FIGS. 3A and 3B, filled in the channel 2 of the microdevice and the bead is preferably larger than the channel width so as to prevent the bead from leaking out from the channel 2. Also, a channel 2c made narrower than the bead diameter may be provided in a part of the channel 2. In the case of using a bead smaller than the width of the channel 2, a structure such as weir or mesh may be provided so that the beads can stay in the channel. The portion in which the beads are filled is preferably broader in the longitudinal or transverse direction than the normal channel. When the beads are received as the nucleic acid-adsorbing support in the microdevice, the surface area is increased and contact with various solutions can be attained with high probability. Accordingly, the adsorption ability can be freely adjusted and the channel can be made very compact without causing reduction in the recovery efficiency due to fabrication as a microdevice.

The case where the channel of the microdevice is made of a nucleic acid-adsorbing support is described below. The function of adsorbing a nucleic acid can be introduced by using a material having a function of adsorbing a nucleic acid as the material constituting the channel or by treating or coating the material constituting the channel.

The length of the channel is preferably from 10 to 500 mm, more preferably from 50 to 200 mm. When the length of the channel is in this range, a sufficiently large contact area is ensured between the nucleic acid contained in the sample solution and the nucleic acid-adsorbing support, and the microdevice can be advantageously produced without passing through a step of filling beads, a filter or the like. Also, within this range, the extraction efficiency does not decrease and this is preferred. The length of the channel can be appropriately adjusted according to the amount or concentration of the nucleic acid contained in the sample solution.

Furthermore, in the channel of the microdevice, a nucleic acid-adsorbing structure may be present as the nucleic acid-adsorbing support. The nucleic acid-adsorbing structure can be constructed by the method described, for example, in Biochip: Iryo wo Kaeru MicroNanotechnolgy Koen Yokoshu (Biochip: Preprint of Lecture on MicroNano-technology of Changing the Medical Treatment), pp. 28-29. This method has been developed for use in electrophoresis of separating a nucleic acid, but the present inventors have found that a nucleic acid can be also extracted by the method. In this method, a nanosize pillar can be continuously produced in the channel by using electron-beam exposure. The surface of the produced pillar may be coated by the same method as described above or a material having a function of adsorbing a nucleic acid may be used as the material constituting the channel.

The nucleic acid-adsorbing structure in the channel is not particularly limited in the form, but the diameter of one structure (pillar) is preferably from 0.1 to 10 μm, and the height (length of pillar) is preferably a size within the depth of the channel. At least two or more structures are preferably produced to continuously exist at intervals of 10 μm or less in the channel. More preferably, the diameter of one structure is from 0.2 to 0.5 μm, the height is from 3 to 100 μm and, for example, in the case of producing the structure to exist in a channel having a regular square tube form of 100 μm×100 μm, the structures are continuously present at intervals of 0.2 to 0.5 μm over the region of 3 to 10 mm in the length direction of the channel.

When a nucleic acid-adsorbing structure is present as the nucleic acid-adsorbing support in the channel of the microdevice, the surface area of the nucleic acid-adsorbing support can be broadened and the contact area with various solutions can be advantageously increased. Also, the channel can be made very compact without causing reduction in the recovery efficiency due to fabrication as a microdevice. Furthermore, in the production of the microdevice, the types of members can be decreased (for example, the microdevice can be produced by using a pair of upper and lower two chips) to ensure excellent production suitability and this is preferred.

<Method for Separating and Purifying Nucleic Acid>

The microdevice of the present invention is used for performing a method for separating and separating a nucleic acid, the method comprising the following steps:
(A) a step of bringing a nucleic acid-containing sample solution into contact with a nucleic acid-adsorbing support having a function of adsorbing a nucleic acid,
(B) a step of washing the nucleic acid-adsorbing support with a washing solution in the state of a nucleic acid being adsorbed to the support, and
(C) a step of desorbing the nucleic acid from the nucleic acid-adsorbing support with use of a recovering solution, thereby purifying the nucleic acid.

The microdevice comprises, as describe above, at least one or more opening and one or more channel for passing a sample solution.

The channel may be one channel or may be branched into two or more channels as long as the steps (A), (B) and (C) above can be performed. For example, the channel for a residual solution after contacting a sample solution with the nucleic acid-adsorbing support and effecting the adsorption of a nucleic acid, the channel for a washing solution after washing the nucleic acid-adsorbing support, and the channel for a recovering solution after desorbing the nucleic acid from the nucleic acid-adsorbing support may be separately provided. Also, the channel may take any form such as linear or curved.

The microdevice comprises one or more opening. The sample solution, washing solution and recovering solution may be injected or discharged through the same opening, or one or more other opening may be provided to discharge these solutions through the opening different from the opening used for injection.

In the microdevice of the present invention, the step from the first injection of a nucleic acid-containing sample solution until the collection of a nucleic acid outside the microdevice can be completed within 20 minutes and in a suitable state, within 1 minute.

In the microdevice of the present invention, a nucleic acid having a molecular weight over a wide range from 1 to 300 kbp, particularly from 20 to 300 kbp, can be recovered. That is, as compared with the conventionally employed spin column method using a glass filter, a long-chained nucleic acid can be recovered.

Also, a nucleic acid having a purity such that the measured value (260 nm/280 nm) by an ultraviolet-visible spectrophotometer is from 1.6 to 2.0 in the case of DNA and from 1.8 to 2.2 in the case of RNA can be recovered, and a high-purity nucleic acid with impurities mingled being in a small amount can be constantly obtained. Furthermore, a nucleic acid having a purity such that the measured value (260 nm/280 nm) by an ultraviolet-visible spectrophotometer is around 1.8 in the case of DNA and around 2.0 in the case of RNA can be recovered.

<Test Sample>

A test sample to be used in the invention is not limited as long as a test sample contains nucleic acid, for examples thereof in the field of diagnostics include body fluids collected as test samples, such as whole blood, plasma, serum, urine, faeces, semen and saliva, or plants (or a part thereof), animals (or a part thereof), bacteria, virus, cultured cells, solutions prepared from biological materials such as lysates and homogenates of the above samples.

The nucleic acid-containing sample may be a sample containing a single nucleic acid, or may be a sample containing different, plural kinds of nucleic acids. Nucleic acids to be recovered are not limited as to kind, and may be DNA or RNA, single-stranded chain or double-stranded chain, and straight or cyclic. The number of samples may be one or plural (parallel treatment of plural samples using plural vessels). The length of nucleic acid to be recovered is not particularly limited, either, and a nucleic acid of any length between, for example, from several bp to several Mbp can be used. In view of handling convenience, the length of a nucleic acid to be recovered is generally from about several bp to about several hundreds kbp. The method of the invention for separation and purification of nucleic acid enables one to recover a comparatively longer nucleic acid expeditiously than that obtained by the conventional simple method for separation and purification of nucleic acid, and can be employed for recovering a nucleic acid of preferably from 20 kbp to 300 kbp, more preferably from 50 kbp to 200 kbp, still more preferably from 70 kbp to 140 kbp.

The nucleic acid recovered may be a single strand or a double strand.

<Pretreatment Step>

The test sample is preferably mixed with a solution (nucleic acid-solubilizing reagent) containing a reagent of solubilizing a nucleic acid by dissolving the cell membrane, nuclear membrane or the like and then uniformized, whereby the cell membrane and the nuclear membrane are dissolved, as a result, the nucleic acid is dispersed in the solution and a nucleic acid-containing sample solution is obtained. This step is called a pretreatment step.

For example, when the objective test sample is a whole blood, A. removal of red blood cell, B. removal of various proteins, and C. dissolution of white blood cell and dissolution of nuclear membrane are preferably performed, because A. removal of red blood cell and B. removal of various proteins can prevent nonspecific adsorption to the nucleic acid-adsorbing support and clogging of the nucleic acid-adsorbing support, and C. dissolution of white blood cell and dissolution of nuclear membrane can solubilize a nucleic acid which is an object of extraction.

The pretreatment step consists of the following steps:
(a) a step of contacting and mixing a test sample (including cell or virus) with a nucleic acid-solubilizing reagent (a solution containing at least any one of a chaotropic salt, a surfactant, a protease, an antifoaming agent and a nucleic acid stabilizer);
(b) a step of adding a water-soluble organic solvent to the mixed solution (solution a) obtained above, and
(c) a step of stirring the mixed solution (solution b) after the addition of an organic solvent.

Before the pretreatment step, a step of homogenizing the test sample in advance (hereinafter sometimes referred to as a “homogenizing step”) is preferably performed. By this treatment, the suitability for automation can be enhanced. The homogenization may be performed, for example, by an ultrasonic treatment, a treatment using a sharp protrusion, a treatment using high-speed stirring, a treatment of extruding the test sample from fine voids, or a treatment using glass beads.

The homogenizing step and the pretreatment step consisting of (a) to (c) may be performed in the microdevice and this is preferred in view of suitability for automation.

The homogenizing step can be performed by disturbing the laminar flow in the channel of the microdevice to cause a turbulent flow. For disturbing the laminar flow, it is preferred to provide a structure in the channel, change the cross-sectional shape of channel, or dispose a substance such as glass bead, but the present invention is not limited thereto.

The pretreatment step may also be performed by providing an opening part for injecting a nucleic acid-solubilizing reagent or a water-soluble organic solvent (the nucleic acid-solubilizing reagent and the water-soluble organic solvent may be injected from the same opening part or may be injected from respective opening parts by providing different opening parts therefor) in the microdevice, and appropriately injecting a nucleic acid-solubilizing reagent or a water-soluble organic solvent along with the transfer of test sample in the microdevice. In the case of using the same opening part, the pretreatment may be performed by providing a liquid reservoir at a position between the opening part and the nucleic acid-adsorbing support, first mixing and stirring a nucleic acid-solubilizing reagent and a sample, reserving the mixed solution in the liquid reservoir, then injecting an organic solvent, and performing mixing and stirring.

Alternatively, the microdevice may be previously imparted with a mechanism such that a contact part of contacting an test sample with a nucleic acid-solubilizing reagent and an addition part of adding a water-soluble organic solvent to the mixed solution obtained in (a) are provided, a nucleic acid-solubilizing reagent and a water-soluble organic solvent are charged into the contact part and the addition part, respectively, and the test sample is injected into the microdevice and while flowing, allowed to reach the contact part or the addition part and be mixed with the nucleic acid-solubilizing reagent or the water-soluble organic solvent. In this case, the test sample is contacted with the nucleic acid-solubilizing reagent and mixed in the course of transfer within the microdevice to give a mixed solution (solution a). Similarly, after a water-soluble organic solvent is added to the mixed solution (solution a), the solution is stirred in the course of transfer within the microdevice to give a mixed solution (solution b). These mechanisms are described by way of example and the present invention is not limited thereto.

{Nucleic Acid-Solubilizing Reagent}

As for the nucleic acid-solubilizing reagent, a solution containing at least any one of a chaotropic salt, a surfactant, a protease, an antifoaming agent and a nucleic acid stabilizer may be used.

(Chaotropic Salt)

Examples of the chaotropic salt which can be used include guanidine salts (e.g., guanidine hydrochloride, guanidine thiocyanate), sodium isothiocyanate, sodium iodide and potassium iodide. Among these, guanidine hydrochloride is preferred. These salts may be used individually or in combination of two or more thereof. The chaotropic salt concentration in the nucleic acid-solubilizing reagent is preferably 0.5 mol/L or more, more preferably from 0.5 to 4 mol/L, still more preferably from 1 to 3 mol/L.

In place of the chaotropic salt, urea may also be used as a chaotropic substance.

(Surfactant)

Surfactants, for example, include a nonionic surfactant, a cationic surfactant, an anionic surfactant, an amphoteric surfactant.

In the invention, the nonionic surfactant and the cationic surfactant can be preferably used.

Nonionic surfactants include a polyoxyethylene alkyl phenyl ether-based surfactant, a polyoxyethylene alkyl ether-based surfactant, and fatty acid alkanolamide, and the preferable one is a polyoxyethylene alkyl ether-based surfactant. Among the polyoxyethylene (POE) alkyl ether surfactant, POE decyl ether, POE lauryl ether, POE tridecyl ether, POE alkylenedecyl ether, POE sorbitan monolaurate, POE sorbitan monooleate, POE sorbitan monostearate, tetraoleic polyoxyethylene sorbit, POE alkyl amine, and POE acetylene glycol are more preferred.

Cationic surfactants include cetyl trimethyl ammonium bromide, dodecyl trimethyl ammonium chloride, tetradecyl trimethyl ammonium chloride, cetyl pyridinium chloride.

These surfactants can be used alone or in combinations of two or more. The concentration of the surfactant in the nucleic acid-solubilizing reagent is preferably from 0.1 to 20% by weight.

(Protease)

As such protease, at least one protease selected from among serine protease, cysteine protease, metal protease, etc. can preferably be used. Also, a mixture of plural kinds of proteases may preferably be used.

The nucleic acid-solubilizing reagent preferably contains a protease in terms of the improvement of the recovering amount and the recovering efficiency of nucleic acid, the significant reduction of the necessary amount of the test sample containing nucleic acid and the rapid operation.

Serine protease is not particularly limited and, for example, protease K can preferably be used. Cysteine protease is not particularly limited and, for example, papain and cathepsin may preferably be used.

Metal protease is not particularly limited and, for example, carboxypeptidase may preferably be used.

The protease can be used, upon addition, in an amount of preferably from 0.001 IU to 10 IU, more preferably from 0.01 IU to 1 IU, per ml of the whole reaction system.

Also, as the protease, a protease not containing nuclease can preferably be used. Also, a protease containing a stabilizing agent can preferably be used. As the stabilizing agent, a metal ion can preferably be used. Specifically, magnesium ion is preferable, and can be added in the form of, for example, magnesium chloride. Incorporation of a stabilizing agent for a protease enables one to reduce the amount of protease necessary for recovery of nucleic acids to a slight amount, which serves to reduce the cost required for recovery of nucleic acids. The amount of the stabilizing agent for protease is preferably from 1 to 1000 mmol/L, more preferably from 10 to 100 mmol/L, based on the whole amount of the reaction system.

The protease may be used as one reagent obtained by previously mixing with other reagents such as a chaotropic salt and a surfactant, thus being used for recovery of nucleic acids.

Alternatively, the protease may be used as a separate reagent from other reagents such as a chaotropic salt and a surfactant.

In the latter case, a sample is first mixed with a reagent containing a protease, and the mixture is then mixed with a reagent containing a chaotropic salt and a surfactant. Or, the protease may be mixed after first mixing a sample with the reagent containing a chaotropic acid and a surfactant.

Also, it is possible to dropwise add from a container retaining a protease directly like an eye lotion to a sample or a mixture of a sample and a reagent containing a chaotropic salt and a surfactant. In this case, operation can be simplified.

(Defoaming Agent)

As the defoaming agent, a silicon-based defoaming agent (e.g., silicon oil, dimethyl polysiloxane, silicon emersion, denatured polysiloxane, silicon compound, etc.), alcohol-based defoaming agent (e.g., acetylene glycol, heptanol, ethyl exanol, superhigh grade alcohol, polyoxy alkylene glycol, etc.), ether-based defoaming agent (e.g., heptyl cellosolve, nonyl cellosolve-3-heptylcorbitol, etc.), fatty oil-based defoaming agent (e.g., animal and plant fat, etc.), fatty acid-based defoaming agent (e.g., stearic acid, oleic acid, palmitic acid, etc.), metallic soap-based defoaming agent (e.g., aluminum stearate, calcium stearate, etc.), fatty acid ester-based defoaming agent (e.g., a natural wax, tributyl phosphate, etc.), phosphate ester-based defoaming agent (e.g., sodium octyl phosphate, etc.), amine-based defoaming agent (e.g., diamyl amine, etc.), amide-based defoaming agent (e.g., amide stearate, etc.), and other defoaming agents (e.g., ferric sulfate, bauxite, etc.) can be exemplified. These defoaming agent can be used alone or in combinations of two or more. Two compounds combined from silicon-based and alcohol-based defoaming agents are especially preferred.

The concentration of a defoaming agent in nucleic acid-solubilizing reagent is preferably in a range of 0.1 to 10% by weight.

(Nucleic Acid Stabilizing Agent)

As the nucleic acid stabilizing agent, one having a reaction to inactivate a nuclease activity can be exemplified. Depending on a test sample, there are cases where nuclease, which degrades nucleic acid, is comprised thereto so that when nucleic acid is homogenized, nuclease reacts with nucleic acid, so as to result in a remarkable reduction of a yield amount. For the purpose of avoiding this, a stabilizing agent having a function to inactivate nuclease can be coexisted in a nucleic acid-solubilizing solution. As a result, improvements in a recovering yield and a recovering efficiency of nucleic acid lead to the minimization and acceleration of a test sample.

As the nucleic acid stabilizing agent having functions to inactivate the nuclease activity, a compound used routinely as a reducing agent can be used. Examples of reducing agents include hydrogenated compounds such as a hydrogen atom, hydrogen iodide, hydrogen sulfide, aluminum lithium hydride, and sodium borohydride; a highly electropositive metal such as alkaline metal, magnesium, calcium, aluminum, and zinc, or their amalgam; organic oxides such as aldhyde-based, sugar-based, formic acid, and oxalic acid; and mercapto compounds. Among these, the mercapto compounds are preferable. Examples of mercapto compounds include N-acetyl cysteine, mercapto ethanol, and alkyl mercaptane or the like.

The concentration of the nucleic acid stabilizing agent in the nucleic acid-solubilizing reagent is preferably from 0.1 to 20% by weight, and more preferably from 0.5 to 15% by weight.

(Water-Soluble Organic Solvent)

The nucleic acid-solubilizing reagent may contain a water-soluble organic solvent. This water-soluble organic solvent is used for the purpose of enhancing the solubility of various reagents contained in the nucleic acid-solubilizing reagent. Examples of the water-soluble organic solvent include acetone, chloroform and dimethylformamide. Among these, alcohol is preferred. The alcohol may be any one of a primary alcohol, a secondary alcohol and a tertiary alcohol. In particular, methanol, ethanol, propanol or an isomer thereof, and butanol or an isomer thereof are more preferred. These water-soluble organic solvents may be used individually or in combination of two or more thereof. The concentration of the water-soluble organic solvent in the nucleic acid-solubilizing reagent is preferably from 1 to 20 weight %.

The nucleic acid-solubilizing reagent solution described above has a pH of preferably 5 to 10, more preferably 6 to 9, still more preferably 7 to 8.

{Mixing}

The method for mixing the test sample (preferably homogenized test sample) with the nucleic acid-solubilizing reagent containing at least any one of a chaotropic salt, a surfactant, a protease, an antifoaming agent and a nucleic acid stabilizer is not particularly limited. At the mixing, the test sample and the nucleic acid-solubilizing reagent are preferably mixed by means of a stirring device at 30 to 3,000 rpm for 1 second to 3 minutes. By this operation, the yield of a nucleic acid separated and purified can be advantageously increased. It is also preferred to effect the mixing by performing rollover mixing from 5 to 30 times. Also, the mixing may be effected by performing a pipetting operation from 10 to 50 times and in this case, the yield of a nucleic acid separated and purified can be increased by a simple and easy operation.

The homogenizing step can also be performed in the microdevice as described above, and the mixing in the microdevice may be effected by disturbing the laminar flow in the channel of the microdevice and thereby causing a turbulent flow.

{Addition of Water-Soluble Organic Solvent}

Subsequently, a water-soluble organic solvent is preferably added to the mixed solution obtained by mixing the test sample (preferably, homogenized test sample) and the nucleic acid-solubilizing reagent. As for the water-soluble organic solvent added to the mixed solution, an alcohol may be used. The alcohol may be any one of a primary alcohol, a secondary alcohol and a tertiary alcohol, and is preferably methanol, ethanol, propanol or an isomer thereof, or butanol or an isomer thereof. The final concentration of such a water-soluble organic solvent in the sample solution containing a nucleic acid is preferably from 5 to 90 weight %.

<Washing and Washing Step>

The washing step (B) and the washing solution are described below. By performing the washing, the amount recovered and the purity of a nucleic acid are enhanced and the amount of a test sample containing the necessary nucleic acid can be rendered extremely small. Also, by automating the washing or recovery operation, the operation can be simply and swiftly performed. The washing step may be completed by once washing and this is preferred because the step can be more expedited. Also, washing may be repeated multiple times and this is preferred because a high-purity nucleic acid can be obtained.

For the transfer of the washing solution in the channel, any transfer system described for the sample solution in the item of <Microdevice> can be used.

In the washing step, the liquid temperature of the washing solution is preferably from 4 to 70° C., more preferably room temperature. Also, in the washing step, stirring by mechanical vibration or ultrasonic wave may be applied to the microdevice simultaneously with the washing step.

In the washing step, the washing solution is a solution containing at least one of water-soluble organic solutions and water-soluble salts is preferred. It is necessary for a washing solution to have ability that works to wash out impurities of the nucleic acid mixture solution, which are adsorbed onto the nucleic acid-adsorbing porous membrane along with nucleic acid. In this regard, the washing solution must have such a composition that it desorbs only impurities from the nucleic acid-adsorbing porous membrane, and not the nucleic acid. In the purpose, nucleic acid are very insoluble to water-soluble organic solvents such as alcohol, therefore the water-soluble organic solvent is suitable for desorbing other substances by maintaining nucleic acid. In addition, adding water-soluble salts enables to increase an adsorption effect of nucleic acid, thereby improving the selectively removing operation for impurities and unnecessary substances.

With regard to a water-soluble organic solvent to be contained in a washing solution, alcohol and acetone etc. can be used, and preferably alcohol is used. As alcohol, methanol, ethanol, propanol and its isomers such as isopropanol, n-propanol, butanol and its isomers etc. may be used and, among them, it is preferred to use ethanol. Amount of the water-soluble organic solvent contained in the washing solution is preferably 20 to 100% by weight and, more preferably, 40 to 80% by weight.

On the other hand, for the water-soluble salt contained in a washing solution, a halide salt is preferred and among them, a chloride salt is more preferred. Further, the water-soluble salt is preferably a monovalent or divalent cation, particularly an alkali metal and an alkali earth metal is preferred. And among them, a sodium salt and a potassium salt are most preferred. When the water-soluble salt is contained in the washing solution, the concentration thereof is preferable 10 mmol/L or more, and the upper limit is not particularly limited as long as the upper limit does not affect solubility of the impurities, 1 mol/L or less is preferred and 0.1 mol/L or less is more preferred. Above all, that the water-soluble salt is sodium chloride and sodium chloride is contained in 20 mmol/L or more and 0.1 mol/L or less is particularly preferred.

In addition, the washing solution is characterized in that a chaotropic substance is not contained therein. As a result, a possibility of having the chaotropic substance incorporated into a recovery step after the washing step can be reduced. In the recovery step, where the chaotropic substance is incorporated thereinto, it sometimes hinders an enzyme reaction such a PCR reaction or the like, therefore considering the afterward enzyme reaction, not including the chaotropic substance to a washing solution is ideal. Further, the chaotropic substance is corrosive and harmful, in this regard, it is extremely advantageous from an operational safety standpoint for the researcher not to use the chaotropic substance when unnecessary.

Herein, the chaotropic substance represents aforementioned urea, guanidine salt, sodium isothiocyanate, sodium iodide, potassium iodide, etc.

Conventionally, in the separation and purification method of a nucleic acid, the washing solution often remains in the channel at the washing step due to high wettability of the washing solution to the channel or the like, and the washing solution is mixed into the recovery step subsequent to the washing step, giving rise to reduction in the purity of a nucleic acid or reduction in the reactivity at the next step. Accordingly, it is important that a residual washing solution does not remain inside the microdevice and the solution used at the adsorption or washing, particularly, the washing solution, does not affect the next step.

Accordingly, in order to prevent contamination of the recovering solution of the subsequent step with the washing solution of the washing step and thereby to keep residue of the washing solution in the cartridge to the minimum, it is desirable that surface tension of the washing solution is less than 0.035 J/m². When the surface tension is low, wettability of the washing solution for the cartridge is improved and volume of the residual solution can be controlled.

Conventionally, in the separation and purification method of a nucleic acid, the washing solution is often scattered and attaches to others and this causes a problem of contamination of the sample. As regards this kind of contamination in the washing step, the microdevice is a closed system and therefore, is advantageous also in that contamination from outside scarcely occurs. Furthermore, contamination in the inside can be prevented by devising means of not causing mixing of the washing solution and the recovering solution, for example, by providing the channels for the washing solution and the recovering solution independently from each other.

<Recovering Solution and Recovery Step>

The recovery step (C), the recovering solution and the recovery container are described below.

The recovering solution is supplied to the nucleic acid-adsorbing support in the microdevice. The recovering solution can be transferred in the channel while contacting it with the nucleic acid-adsorbing support.

For the transfer of the recovering solution in the channel, any transfer system described for the sample solution in the item of <Microdevice> can be used.

As for the recovering solution, for example, purified distilled water or Tris/EDTA buffer can be used. The pH of the recovering solution is preferably from 2 to 11, more preferably from 5 to 9. The recovering solution is preferably a solution having a salt concentration of 0.5 mol/L or less. In particular, the ion intensity and the salt concentration affect the elution of the adsorbed nucleic acid. The ion intensity of the recovering solution is preferably 0.5 mol/L or less, more preferably 290 mmol/L or less. Within such a range, the recovery percentage of a nucleic acid is elevated and a larger amount of a nucleic acid can be recovered. Furthermore, in the case of using the recovered nucleic acid for PCR (polymerase chain reaction), the buffer solution (for example, an aqueous solution having a final concentration that KCl is 50 mmol/L, Tris-HCl is 10 mmol/L and MgCl₂ is 1.5 mmol/L) for use in the PCR reaction may be used as the recovering solution. When a buffer solution suitable for the PCR method is used, transition to the PCR step after recovery can be easily and swiftly performed.

When volume of a recovering solution is made small as compared with the initial volume of a sample solution containing nucleic acid, it is now possible to prepare a recovered solution containing concentrated nucleic acid. Preferably, the ratio of (volume of recovering solution):(volume of sample solution) is able to be made 1:100 to 99:100 and, more preferably, it is able to be made 1:10 to 9:10. As a result thereof, nucleic acid is now able to be easily concentrated without conducting an operation for concentrating in a step after separation and purification of nucleic acid. According to such a method, a method for producing a nucleic acid solution in which nucleic acid is concentrated as compared with a test body is able to be provided.

Another method is that desorption of nucleic acid is conducted under a condition where volume of a recovering solution is more than the initial volume of a sample solution containing nucleic acid whereby it is possible to prepare a recovering solution containing nucleic acid of a desired concentration and to prepare a recovering solution containing nucleic acid which is suitable for the next step (such as PCR). Preferably, the ratio of (volume of recovering solution):(volume of sample solution) is able to be made 1:1 to 50:1 and, more preferably, it is able to be made 1:1 to 5:1. As a result thereof, there is an advantage that, after separation and purification of nucleic acid, troublesomeness for adjustment of concentration is no longer necessary. In addition, as a result of use of a sufficient amount of a recovering solution, an increase in a recovering rate of nucleic acid from the porous membrane is able to be achieved.

The nucleic acid can be easily recovered by changing the temperature of the recovering solution according to the purpose. For example, the nucleic acid is preferably desorbed from the support by adjusting the temperature of the recovering solution to 0 to 10° C., because the activity of a nucleolytic enzyme can be suppressed without adding any reagent or special operation for preventing decomposition by the enzyme, as a result, decomposition of the nucleic acid can be avoided and a nucleic acid solution can be easily and simply obtained with good efficiency.

Also, when the temperature of the recovering solution is adjusted to 10 to 35° C., the nucleic acid can be recovered at room temperature in general and further be separated and purified by desorbing the nucleic acid without requiring any complicated step and this is preferred.

In another method, the temperature of the recovering solution is adjusted to a high temperature, for example, from 35 to 70° C., whereby desorption of a nucleic acid from the support can be simply and easily performed at a high recovery percentage without passing through a cumbersome operation.

There is no limitation for the infusing times for a recovering solution and that may be either once or plural times. Usually, when nucleic acid is to be separated and purified quickly and simply, that is carried out by means of one recovery while, when a large amount of nucleic acid is to be recovered, recovering solution may be infused for several times.

Also, in the recovering step, it is possible to add a stabilizing agent for preventing degradation of nucleic acid recovered in the recovering solution of nucleic acid. As the stabilizing agent, an antibacterial agent, a fungicide, a nucleic acid degradation inhibitor and the like can be added. As the nuclease inhibitor, EDTA and the like can be cited. In addition, as another embodiment, a stabilizer can also be added to the recovery container in advance.

Also, the recovery container to be used in the recovery step is not particularly limited, a recovery container prepared from a raw material having no absorption at 260 nm can be used. In that case, concentration of the recovered RNA solution can be measured without transferring it into other container. As the raw material having no absorption at 260 nm, quartz glass and the like can for example be used, though not limited thereto.

As for the step next to the recovery step, a PCR amplifying step is sometimes performed. The PCR amplifying step may be practiced within the microdevice. In this case, the microdevice is required to have a channel for injecting a reagent for the PCR amplification and/or a channel for stirring it and furthermore, a device for controlling the temperature is necessary.

In the microdevice, the method for separating and purifying a nucleic acid may also be performed by using a device.

The reagents for use in the microdevice may be prepared as a reagent kit. The reagent kit contains the nucleic acid-solubilizing agent, the washing solution and the recovering solution.

Also, these reagents and/or the water-soluble organic solvent may be previously held in the microdevice.

### EXAMPLES

The present invention is described in greater detail below by referring to Examples, but the present invention is not limited thereto.

### Example 1

### Microdevice Receiving Nucleic Acid-Adsorbing Porous Membrane as Nucleic Acid-Adsorbing Support

(1)-1. Production of Device for Separating and Purifying Nucleic Acid

As shown in FIG. 1A, in the center of a PDMS (polydimethylsiloxane)-made flat plate 1 with a size of 10 mm×10 mm×3 mm, a vertical channel 2 with an inner diameter of 500 μm was produced to prepare chips 4a and 4b shown in FIG. 1B. As shown in FIG. 1B, a nucleic acid-adsorbing porous membrane 3 with a diameter of 1 mm was sandwiched by two chips 4a and 4b, and these were press-bonded under heat to produce a microdevice shown in FIG. 1C. In the above, a regenerated cellulose was used as the nucleic acid-adsorbing porous membrane 3.

(1)-2. Preparation of Nucleic Acid-Solubilizing Reagent and Washing Solution

A nucleic acid-solubilizing reagent solution and a washing solution according to the formulation shown in Table 1 were prepared.

| | | |
|---|---|---|
| (Nucleic Acid-Solubilizing Reagent Solution) | | |
| Guanidine hydrochloride (produced by Life | 382 | g |
| Technologies, Inc.) | | |
| Tris (produced by Life Technologies, Inc.) | 12.1 | g |
| Triton X-100 (produced by ICN) | 10 | g |
| Distilled water | 1,000 | ml |
| (Washing Solution) | | |
| 100 mM NaCl | | |
| 10 mM Tris-HCl | | |
| 65% Ethanol | | |

(1)-3. Operation for Separating and Purifying DNA

The nucleic acid-solubilizing reagent (10 μl) prepared above and 1 μl of a protease (“Protease” Type XXIV Bacterial, produced by SIGMA) solution were added to 10 μl of a human whole blood test sample and incubated at 60° C. for 10 minutes. After the incubation, 10 μl of ethanol was added and stirred to produce a nucleic acid-containing sample solution. This nucleic-acid containing sample solution was injected into the opening 2a of the microdevice with a nucleic acid-adsorbing porous membrane 3 produced in (1)-1 above and subsequently, the washing solution prepared in (1)-2 above was passed with intervention of a fixed amount of air and discharged from the opening 2b. After thorough passing of the washing solution, a recovering solution having an ion intensity of 10 mmol/L was similarly flowed with intervention of a fixed amount of air, passed through the porous membrane 3 and discharged from the opening 2b, and this solution was recovered. As for the means of transporting the sample solution, washing solution and recovering solution in the channel, the liquid droplet (liquid plug) system was used. FIG. 2 is a schematic explanatory view describing the step of passing liquids by using a pressure-applying pump 5 as an external pressure source.

(1)-4. Amplification of PCR

Using the nucleic acid purified in (1)-3 above, amplification of nucleic acid by the polymerase chain reaction was performed.

The reaction solution of PCR was prepared from purified water (36.5 μL), 10×PCR buffer (5 μL), 2.5 mM dNTP (4 μL), Taq FP (0.5 μL), primer (2 μL) and nucleic acid solution (2 μL).

In the PCR, one cycle was consisting of alteration at 94° C. for 30 seconds, annealing at 65° C. for 30 seconds and elongation at 72° C. for 1 minute, and 30 cycles were repeated.

Also, Human DNA produced by Clontech was used as the positive control.

The following primer was used.

p53 Exon6:

| | |
|---|---|
| Forward: | GCGCTGCTCA GATAGCGATG |
| Reverse: | GGAGGGCCAC TGACAACCA |

### Example 2

### Microdevice Receiving Bead as Nucleic Acid-Adsorbing Support

(2)-1. Production of Nucleic Acid-Adsorbing Bead

Polystyrene-made beads of φ=10 μm were dispersed in a methylene chloride solution of triacetylcellulose and dried. The dried beads were washed with water, dispersed in an aqueous 0.4 mol/L NaOH solution, stirred at room temperature for 30 minutes, filtered and again thoroughly washed with water.

(2)-2. Preparation of PDMS Concave Mold

SU-8 which is a thick-film photoresist was spin-coated on a silicon wafer to a film thickness of 100 μm and after preheating at 90° C. for 1 hour, irradiated with UV light through a mask (not shown) having a channel pattern corresponding to FIG. 3A, and the portion irradiated with light was cured at 90° C. for 1 hour. The uncured portion was dissolved and removed with propylene glycol monomethyl ether acetate (PGMEA) and after washing with water and drying, the wafer was used as a silicon wafer/SU8 convex mold.

Subsequently, PDMS (a 10/1 mixed solution of DuPont Sylgard/curing solution) was cast on the silicon wafer convex mold, cured at 80° C. for 2 hours and then gently peeled off from the silicon wafer convex mold to produce a PDMS concave mold 7 shown in FIG. 3A.

The mold was adjusted such that the injection port 8 and recovery port 9 each had a diameter of 1 mm, the waste liquor port 10 had a diameter of 2 mm, the channel 2 had a width of 200 μm, and the depth was 80 μm in any portion.

In a part of the end portion of the thus-produced channel 2, where the beads were filled, the width was decreased to 5 μm so as to prevent the beads from flowing out (in FIG. 3B, the channel 2c). The distal end was divided into a channel for passing the waste liquor and a channel for passing the recovering solution, the boundary therebetween was designed not to allow for mixing of respective liquids by providing a valve 12, and the waste liquor port 10 at the end of the channel for passing the waste liquor and the recovery port 9 at the end of the channel for passing the recovering solution were connected to a suction generating device (pump 13) to enable adjusting which channel was used.

(2)-2. Operation of Separating and Purifying DNA

The nucleic acid-solubilizing reagent (10 μl) prepared in Example 1 and 1 μl of a protease (“Protease” Type XXIV Bacterial, produced by SIGMA) solution were added to 10 μl of a human whole blood test sample and incubated at 60° C. for 10 minutes. After the incubation, 10 μl of ethanol was added and stirred to produce a nucleic acid-containing sample solution. This nucleic-acid containing sample solution was injected into the injection port 8 of the microdevice receiving the beads 11 produced in (2)-1 above and subsequently, the washing solution prepared in Example 1 was passed with intervention of a fixed amount of air and discharged from the waste liquor port 10. After thorough passing of the washing solution, a recovering solution having an ion intensity of 10 mmol/L was similarly flowed with intervention of a fixed amount of air, passed through the channel 2, thereby contacting the solution with the beads 11, and discharged from the recovery port 9, and this solution was recovered.

(2)-3. Amplification of PCR

The same operation as in (1)-4 was performed except for using the nucleic acid purified in (2)-2.

### Example 3

### Microdevice with Channel Made of Nucleic Acid-Adsorbing Support

(3)-1. Production of Device for Separating and Purifying Nucleic Acid

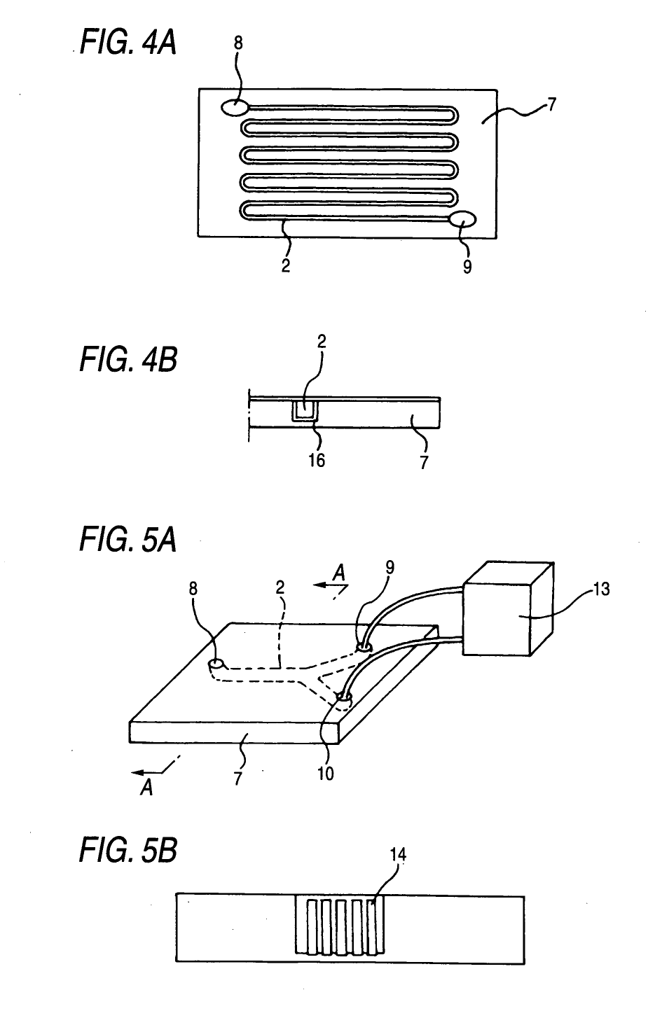

A channel 2 of 100 μm×100 μm was produced to a length of 150 mm for a PDMS (polydimethylsiloxane)-made device with a size of 20 mm×30 mm×3 mm shown in FIG. 4A, in the same manner as in Example 2. The inner side of the channel 2 was, as shown in FIG. 4B, coated with dextrin. A driving system for flowing a liquid from the injection port 8 of the thus-created channel was provided to enable supplying a liquid to the channel and on the other side, a discharge and recovery port 9 allowing for discharge of the liquid and an air vent were provided, thereby producing a microdevice.

(3)-2. Operation of Separating and Purifying DNA

The nucleic acid-solubilizing reagent (10 μl) prepared in Example 1 and 1 μl of a protease (“Protease” Type XXIV Bacterial, produced by SIGMA) solution were added to 10 μl of a human whole blood test sample and incubated at 60° C. for 10 minutes. After the incubation, 10 μl of ethanol was added and stirred to produce a nucleic acid-containing sample solution. This nucleic-acid containing sample solution was injected into the injection port 8 of the device with a channel produced in (3)-1 above and subsequently, the washing solution prepared in Example 1 was passed with intervention of a fixed amount of air and discharged from the recovery port 9. After thorough passing of the washing solution, a recovering solution having an ion intensity of 10 mmol/L was similarly flowed with intervention of a fixed amount of air, passed through the channel 2 and discharged from the recovery port 9, and this solution was recovered. As for the means of transporting the liquids in the channel, the liquid droplet (liquid plug) system was used similarly to (1)-3 above.

(3)-3. Amplification of PCR

The same operation as in (1)-3 was performed except for using the nucleic acid purified in (3)-2.

### Example 4

### Microdevice Having Nucleic Acid-Adsorbing Structure as Nucleic Acid-Adsorbing Support in Channel

(4)-1. Production of Device for Separating and Purifying Nucleic Acid

For a PDMS (polydimethylsiloxane)-made chip with a size of 20 mm×30 mm×3 mm produced in the same manner as in Example 2, as shown in FIG. 5B, structures (nanopillars 14) were provided over a channel length of 15 mm in a channel 2 of 100 μm×100 μm. The nanosize pillars 14 were continuously produced in the channel 2 by using electron-beam exposure according to the method described in Biochip: Iryo wo Kaeru Micro•Nanotechnolgy Koen Yokoshu (Biochip: Preprint of Lecture on Micro•Nanotechnology of Changing the Medical Treatment), pp. 28-29. The nanopillar 14 had a columnar shape with a diameter of 0.2 μm and a height of 100 μm and these pillars were continuously produced at intervals of 0.2 μm. The inner side of the channel was coated with dextrin. The distal end of the channel was divided into a channel for passing the waste liquor and a channel for passing the recovering solution, the boundary therebetween was designed not to allow for mixing of respective liquids by providing a valve 12, and the waste liquor port 10 at the end of the channel for passing the waste liquor and the recovery port 9 at the end of the channel for passing the recovering solution were connected to a suction generating device (pump 13) to enable adjusting which channel was used. In this way, a microdevice for separating and purifying a nucleic acid was produced.

(4)-2. Operation of Separating and Purifying DNA

The nucleic acid-solubilizing reagent (10 μl) prepared in Example 1 and 1 μl of a protease (“Protease” Type XXIV Bacterial, produced by SIGMA) solution were added to 10 μl of a human whole blood test sample and incubated at 60° C. for 10 minutes. After the incubation, 10 μl of ethanol was added and stirred to produce a nucleic acid-containing sample solution. This nucleic-acid containing sample solution was injected into the injection port 8 of the device for separating and purifying a nucleic acid produced in (4)-1 above, in which nucleic acid-adsorbing structures 14 were provided. Subsequently, the washing solution prepared in Example 1 was passed with intervention of a fixed amount of air and discharged from the waste liquor port 10. After thorough passing of the washing solution, a recovering solution having an ion intensity of 10 mmol/L was similarly flowed with intervention of a fixed amount of air, passed through the channel having nucleic acid-adsorbing structures 14, and discharged from the recovery port 9, and this solution was recovered. As for the means of transporting the liquids in the channel 2, the liquid droplet (liquid plug) system was used similarly to (1)-3 above.

(4)-3. Amplification of PCR

The same operation as in (1)-4 was performed except for using the nucleic acid purified in (4)-2.

[Confirmation of Recovery of DNA]

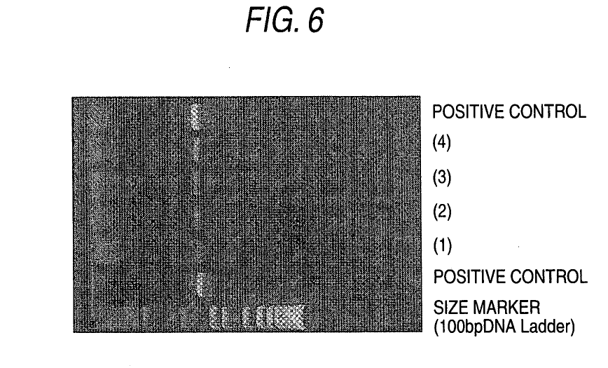

FIG. 6 shows the results of electrophoresis of DNA after separation and purification from the nucleic acid-containing sample solutions obtained in Examples 1 to 4 and amplification by PCR.

From these results, it is seen that in any device, a nucleic acid can be easily and swiftly separated and purified from a nucleic acid-containing sample solution while maintaining the yield and purity.

In any case of Examples 1 to 4, DNA could be separated and purified from the human whole blood within 5 minutes in the operation of separating and purifying DNA.

A microdevice capable of easily and swiftly separating and purifying a nucleic acid from a nucleic acid-containing sample solution can be obtained. Also, a nucleic acid can be easily and swiftly recovered by using the microdevice while maintaining the yield and purity in conventional methods for separating a nucleic acid. Furthermore, with use of an apparatus for using the microdevice or a reagent kit for use in the microdevice, a nucleic acid can be more easily and swiftly recovered.

The entire disclosure of each and every foreign patent application from which the benefit of foreign priority has been claimed in the present application is incorporated herein by reference, as if fully set forth.

What is claimed is:

1. A microdevice for performing a method for separating and purifying a nucleic acid, the microdevice comprising:
at least one opening; and
at least one channel for passing a sample solution,
wherein the method comprises:
(A) a step of bringing a nucleic acid-containing sample solution into contact with a nucleic acid-adsorbing support having a function of adsorbing a nucleic acid;
(B) a step of washing the nucleic acid-adsorbing support with a washing solution in a state of a nucleic acid being adsorbed to the support; and
(C) a step of desorbing the nucleic acid from the nucleic acid-adsorbing support by a recovering solution, thereby purifying the nucleic acid.

2. The microdevice according to claim 1,
wherein the method for separating and purifying a nucleic acid by utilizing the microdevice comprises a pretreatment step of:
mixing a test sample and a nucleic acid-solubilizing reagent, so as to obtain a mixture; and
uniformizing the mixture to obtain a nucleic acid-containing sample solution, and
the microdevice further comprises a mechanism of performing the pretreatment step.

3. The microdevice according to claim 1,
wherein the channel has a width of 1 to 3,000 μm.

4. The microdevice according to claim 1,
wherein the microdevice receives a nucleic acid-adsorbing porous membrane as the nucleic acid-adsorbing support.

5. The microdevice according to claim 1,
wherein the microdevice receives a nucleic acid-adsorbing bead as the nucleic acid-adsorbing support.

6. The microdevice according to claim 1,
wherein the channel comprises a nucleic acid-adsorbing support.

7. The microdevice according to claim 1,
wherein the channel has a nucleic acid-adsorbing structure as the nucleic acid-adsorbing support in the channel.

8. The microdevice according to claim 1,
wherein the sample solution is a solution resulting from adding a water-soluble organic solvent to a solution obtained by treating a test sample with a nucleic acid-solubilizing reagent.

9. The microdevice according to claim 1,
wherein the nucleic acid-solubilizing reagent is a solution containing at least one of a chaotropic salt, a surfactant, a protease, an antifoaming agent and a nucleic acid stabilizer.

10. The microdevice according to claim 1,
wherein the washing solution is a solution containing at least one of methanol, ethanol, propanol or an isomer thereof, and butanol or an isomer thereof in an amount of 20 to 100 weight %.

11. The microdevice according to claim 1,
wherein the recovering solution is a solution having a salt concentration of 0.5 mol/L or less.

12. An apparatus for utilizing a microdevice according to claim 1.

13. A reagent kit for use in a microdevice according to claim 1.
